(19) United States
(12) Plant Patent Application Publication
(10) Pub. No.: US 2003/0009798 P1
(43) Pub. Date: Jan. 9, 2003

(54) Grape plant named 'MN 1166'

(76) Inventors: James Luby, St. Paul, MN (US); Peter Hemstad, Edina, MN (US)

Correspondence Address: Mark S. Ellinger, Ph.D., Fish & Richardson P.C., P.A., Suite 3300, 60 South Sixth Street, Minneapolis, MN, 55402, US

(21) Appl. No.: 09/829,302

(22) Filed: Apr. 9, 2001

(51) Int. Cl. A01H 5/00

(52) U.S. Cl. PLT/205

(57) ABSTRACT

The invention is a new and distinct variety of grapevine designated ‘MN 1166’, which has a combination of outstanding wine quality and cold hardiness.

### BACKGROUND OF THE INVENTION

Grape is one of the most widely planted and economically important fruit crops in the world. Currently, worldwide planting of grapevines exceeds 8.7 million hectares producing 60 million tons annually, with approximately 70% of the grapes being fermented into wine, 28% used for fresh fruit and the remainder used for dried raisins. In the U.S. alone, the grape crop is worth approximately $3 billion annually

Most grape varieties used for production of high quality wines are of the species Vitis vinifera. The discovery of winemaking, as well as the domestication of the wine grape, likely began in southern Caucasia (presently northwestern Turkey, northern Iraq, Azerbaijan and Georgia) at least 6000 years ago. Eventually, varieties of V. vinifera were cultivated throughout the Mediterranean region, then into Europe and Asia, and, ultimately, through European colonization to North America, South America and Australia. On all these continents, successful production of V. vinifera occurs predominantly in temperate climate zones similar to that of the indigenous range of V. vinifera in Eurasia, e.g., summers that are sunny, warm, and dry, and winters that are mild and rainy. V. vinifera varieties cultivated in northern regions of the United States with a continental climate are often subject to serious injury or death from low temperatures during winter. Although several wild Vitis species occur in colder regions of North America and eastern Asia, the wine made from these species generally has serious defects. Thus, there is a need for grape varieties that are winter hardy, yet produce fruit capable of yielding high quality wine.

### BRIEF SUMMARY OF THE INVENTION

‘MN 1166’ is a variety of grape (Vitis sp.) that has a unique combination of outstanding wine quality and very high cold hardiness not found in existing grape varieties. Fruit of ‘MN 1166’ can be fermented to produce white wine having desirable aromas of citrus, apricot, pineapple, and muscat (as found in Riesling or Vignoles varieties) and lacks ‘foxy’ aromas associated with V. labrusca and herbaceous aromas associated with V. riparia. As grown in east central Minnesota, the plants of ‘MN 1166’ plants are moderately vigorous and very winter hardy. The vines are somewhat resistant to phenoxy herbicide injury and moderately susceptible to foliar phylloxera (Daktulospahira vitifoliae) damage. Downy mildew, caused by Plasmopara viticola, has been observed at moderate levels on the foliage, but has not been seen on the fruit. Powdery mildew disease, caused by Uncinula necator, has been seen at low levels on the foliage, but not on the fruit. The disease black rot, caused by Guignardia bidwellii, has been observed sporadically and at low levels on the leaves, but not on the fruit. ‘MN 1166’ vines set a light to moderate crop load that varies from year to year. The fruit are borne on a loose, medium sized cluster. The berries are small to medium sized and amber with a waxy bloom at maturity. In some years, a small percentage of the berries have been observed dropping from the cluster before or during harvest. The berries have not been observed to split, even under wet conditions in the autumn. The fruit at harvest is usually relatively high in sugar and moderate to high in acidity.

### BRIEF DESCRIPTION OF THE DRAWINGS

The accompanying color photograph shows characteristics of ‘MN 1166’ grown under typical field conditions. The photograph depicts color features as true as is reasonably possible.

FIG. 1 is a color photograph showing typical fruit clusters of ‘MN 1166’.

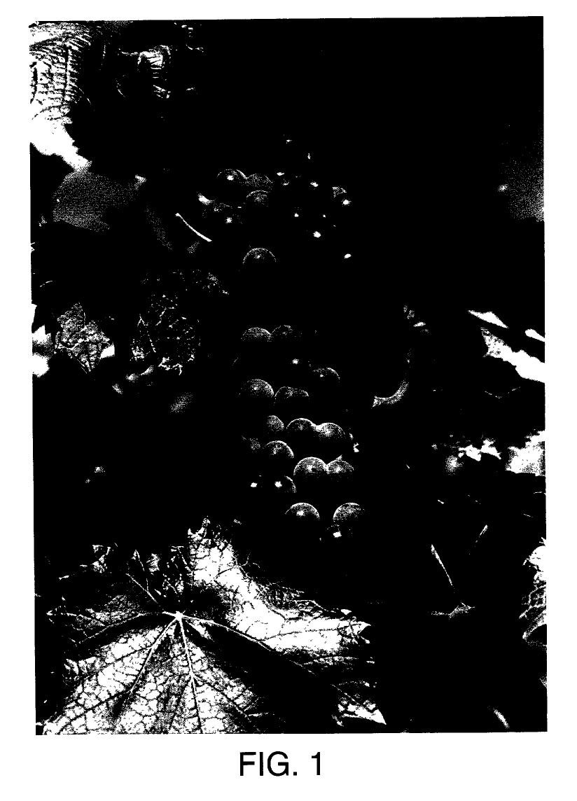

### DETAILED BOTANICAL DESCRIPTION

‘MN 1166’ arose from a controlled cross as part of the grape breeding program at the University of Minnesota Horticultural Research Center (HRC) in Carver County, Minnesota. ‘MN 1166’ was a seedling from the cross designated GE 8824 made in 1988 and having the parentage ‘St. Pepin’×‘E.S. 6-8-25’. The ‘St. Pepin’ variety is described in U.S. Pat. No. PP5,771. The ‘E.S. 6-8-25’ variety is an unnamed selection resulting from a V. riparia ×‘Muscat Hamburg’ cross. ‘MN 1166’ was selected as an initial seedling vine at location Block 1 Row 19 Post 9.9 at the HRC in 1992. ‘MN 1166’ was first asexually propagated by hardwood cuttings in 1993 at the HRC. The variety has been designated ‘MN 1166’.

The following data pertains to vines grown at the University of Minnesota Horticultural Research Center in Carver County, Minnesota near Excelsior. For comparison purposes, data were collected for certain morphological descriptors from plants and fruit of the variety Seyval, a grape variety commonly grown in Minnesota and the eastern U.S. for the production of white wine. Alphanumeric color designations refer to values based on the R.H.S. Colour Chart published by the Royal Horticultural Society, London, England. Many of the descriptors are based on those set forth by the International Board for Plant Genetic Resources in collaboration with the Office Internationale de la Vigne et du Vin (OIV) and the International Union for the Protection of New Varieties of Plants.

When dimensions, sizes, colors and other characteristics are given, it is to be understood that such characteristics are approximations set forth as accurately as possible. Variations of the usual magnitude incident to climatic factors, fertilization, pruning, pest control and other cultural practices are to be expected.

A) Mature Canes The values presented are the means (with ranges in parentheses) of 10 canes observed from the 2000 growing season.

1. Diameter at base.—9.0 cm (7.8-11.7)

2. Diameter at midpoint.—7.1 cm (4.8-8.8)

3. Color of canes.—striated, yellowish brown RHS color chips 175A, 175B, 175C

4. Internode length at base.—2.7 cm (1.9-4.0)

5. Internode length at midpoint.—12.2 cm (7.7-15.3)

6. Lenticels present.—no

7. Cane cross-section shape.—elliptical

8. Density of hairs on mature cane.—very sparse to none

9. Average total cane length.—variable

10. Tendril pattern on shoot.—2,0,2,0 etc. (two nodes with a tendril followed by one node without)

11. Tendrils forked.—yes, one side usually forked twice

12. Tendril texture.—striated

13. Tendril length.—17 cm (12-24)

14. Bud width.—5.3 mm (4.1-6.4)

15. Bud shape.—triangular

B) Mature Leaves Ten representative mature leaves from above the clusters in the middle third of the shoot were examined. The leaves were pressed and dried for later analysis. The values presented below are means (with ranges in parentheses) from collections in September 2000. Descriptors of mature leaves, including the designations Ni through N5, relate to “OIV—Code Numbers 065 -093” of Preliminary Minimal Descriptor List for Grapevine Varieties (Dettweiler E., 1991, Institüit fur Rebenzüchtung, Geilweilerhof, Germany).

| | ‘MN 1166’ | Seyval |
|---|---|---|
| 1. Length of blade: | 13.0 cm (10.1-16.0) | 14.1 cm (12.5-15.0) |
| 2. Width of blade: | 11.5 cm (9.2-13.3) | 12.3 cm (11.1-13.4) |
| 3. Shape of blade: | cuneiform | cordate-circular |
| 4. Number of lobes: | 3.7 (3-5) | 2.7 (0-5) |
| 5. Length of vein N1: | 10.3 cm (8.2-12.1) | 10.0 cm (9.3-11.3) |
| 6. Length of vein N2: | 8.6 cm (6.5-10.1) | 8.5 cm (7.2-10.5) |
| 7. Length of vein N3: | 5.4 cm (3.8-6.8) | 6.1 cm (5.3-7.4) |
| 8. Length of vein N5: | 1.4 cm (0.7-2.3) | 2.6 cm (1.5-3.3) |
| 9. Length of N2 teeth: | 13.4 mm (9-19) | 14.8 mm (9-22) |
| 10. Width of N2 teeth: | 13.7 mm (9-19) | 14.4 mm (9-19) |
| 11. Length/width ratio | | |
| of N2 teeth: | 0.98 (0.8-1.2) | 1.04 (0.8-1.5) |
| 12. Length of N4 teeth: | 6.6 mm (5-9) | 7.2 mm (4-11) |
| 13. Width of N4 teeth: | 8.7 mm (7-10) | 10.8 mm (7-15) |
| 14. Length/width ratio | | |
| of N4 teeth: | 0.76 (0.6-1.0) | 0.67 (0.4-0.9) |
| 15. Shape of teeth: | rectilinear, some | convex |
| | convex | |
| 16. Shape of petiolar sinus: | open | lobes slightly |
| | | overlapping |
| 17. Shape of base | u-shaped | v-shaped |
| of petiolar sinus: | | |
| 18. Depth of petiolar sinus: | 22.1 mm (17-29) | 25.9 mm (18-35) |
| 19. Width of petiolar sinus: | 22.9 mm (14-37) | 10.3 mm (6-15) |
| 20. Length of petioles: | 6.6 cm (4.5-8.6) | 8.8 cm (6.5-11.0) |
| 21. Pubescence on | sparse on veins and | |
| adaxial surface: | petioles, none at | |
| | vein junctions | |
| 22. Pubescence on | abundant at main | |
| abaxial surface: | vein junctions and | |
| | at petiolar junction | |

C) Fruit The values presented below are means (with ranges in parentheses) from fruit observed in the 2000 growing season, except for those traits indicated (**), which are means from the 1995, 1999, and 2000 growing seasons.

| | ‘MN 1166’ | Seyval |
|---|---|---|
| 1. Cluster length: | 14.7 cm (12.5-17.6) | 12.1 cm (9.0-15.1) |
| 2. Cluster weight:** | 86.8 g (53.6-167.9) | 144.2 g (92.4-222.3) |
| 3. Cluster density: | loose | medium |
| 4. Berry weight:** | 1.30 g (1.10-1.66) | 1.90 g (1.59-2.22) |
| 5. Berry length: | 12.8 mm (11.5-14.2) | 13.8 mm (12.2-15.4) |
| 6. Berry diameter at | 12.4 mm (11.5-14.0) | 13.3 mm (12.1-15.3) |
| equator: | | |
| 7. Berry shape: | roundish | roundish |
| 8. Berry cross-section: | circular | circular |
| 9. Berry, color of skin: | yellow-amber | yellow |
| 10. Berry, color of flesh: | no color | no color |
| 11. Berry, particular | apricot | neutral |
| flavor: | | |
| 12. Length of pedicel: | very short | short |
| 13. Berry, separation | | |
| from pedicel: | easy | difficult |
| 14. Berry, presence of | fully developed | fully developed |
| seeds: | | |
| 15. Seed number/berry: | 2.3 (1-4) | 2.2 (1-4) |
| 16. Seed length: | 0.57 mm (0.48-0.65) | 0.59 mm |
| | | (0.54-0.65) |
| 17. Seed width: | 0.31 mm (0.26-0.37) | 0.39 mm |
| | | (0.35-0.46) |
| 18. Seed length/width | 1.84 | 1.51 |
| ratio: | | |
| 19. Seed weight: | 0.020 g | 0.031 g |

D) Harvest Parameters Values represent the means (with ranges in parentheses) for fruit harvested over six growing seasons (1994, 1996 -2000) for ‘MN 1166’ and four growing seasons (1995, 1996, 1999, 2000) for Seyval.

| | ‘MN 1166’ | Seyval |
|---|---|---|
| 1. Harvest date: | 9/26 (9/16-10/5) | 9/27 (9/16-10/6) |
| 2. Brix: | 24.5° (22.6°-27.6°) | 20.9° (18.6°-23.2°) |
| 3. pH: | 3.00 (2.63-3.15) | 3.15 (2.91-3.41) |
| 4. % titratable acidity: | 1.19% (0.93-1.57%) | 0.89% (0.80-1.02%) |

E) Vineyard Performance Based on observations compiled over 9 years (1992 - 2000).

1 Susceptibility to powdery mildew(Uncinula mecator).—low

2 Susceptibility to downy mildew (Plasmopara viticola).—moderate

3 Susceptibility to black rot (Guignardia bidwellii).—low

4 Susceptibility to bunch rot (Botrytis, etc).—very low

5 Susceptibility to foliar phylloxera (Daktulospahira vitifoliae).—moderate

6 Susceptibility to Crown gall (Agrobacterium tumefaciens).—no natural infection observed

7 Susceptibility to phenoxy herbicide drift (e.g., 2,4D).—low

8 Berry splitting.—none observed

9 Berry shelling.—slight to moderate

10. Vigor hardiness.—moderate

11. Winter hardiness.—high, trunks have survived —38° C.

12. Wood ripening.—good

F) Wine Quality Descriptions below are compiled from observations on wine made from ‘MN 1166’ fruit harvested during the 1994 -2000 growing seasons.

1. Flavors and aromas.—apricot, citrus (grapefruit, tangerine), pineapple, muscat; no ‘hybrid’, herbaceous, or labrusca aromas

2. Balance.—good body, well balanced when finished with residual sugar

3. Color.—attractive golden yellow

4. Propensity for oxidation.—low

5. Overall quality.—excellent, reminiscent of Riesling or Vignoles

What is claimed is:

1. A new and distinct variety of grapevine designated ‘MN 1166’ as described and illustrated herein.
